(19) United States
(12) Patent Application Publication
(10) Pub. No.: US 2009/0065000 A1
(43) Pub. Date: Mar. 12, 2009

(54) Endotracheal Tube Stylet and Endotracheal Tube Installation Kit

(76) Inventor: Tien-Sheng Chen, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/934,225

(22) Filed: Nov. 2, 2007

(30) Foreign Application Priority Data
Sep. 7, 2007 (TW) 096215087

(51) Int. Cl. A61M 16/04; A61B 1/267

(52) U.S. Cl. 128/200.26; 600/194; 600/199

(57) ABSTRACT

This invention is to provide an endotracheal tube stylet insertable into an endotracheal tube, the endotracheal tube stylet comprising: a first coupling member; a second coupling member coupled to the first coupling member; an operable member connected to the first or the second coupling member, the operable member being capable of changing the relative positions of the first and the second coupling members; and a turnable member connected to the first or the second coupling member, wherein the turnable member may be redirected by the change of the relative positions of the first and the second coupling members so as to change the shape of the endotracheal tube.

### BACKGROUND OF THE INVENTION

1. Field of the Invention

The present invention relates to an endotracheal tube stylet and more particularly to an endotracheal tube stylet capable of changing the shape of the endotracheal tube.

2. Description of the Related Art

Endotracheal intubation is a critical means for maintaining the breathing function of a patient under general anaesthesia. In most cases, to prevent the occurrence of hypoxia, anaesthetists have to complete the intubation by inserting an endotracheal tube into the patient's trachea in a very short period of time to provide oxygen thereinto promptly. Thus, it is vital that anaesthetists perform the intubation efficiently.

However, due to the structural differences of the upper airways among patients of different ages or sizes, the patients' tracheas sometimes cannot be easily located. Thus, in practice, anaesthetists always have auxiliary tools at hand to facilitate the intubation process. For example, as shown in FIG. 1, an endotracheal tube stylet 10a may be inserted into an endotracheal tube 20 and bent to a specific curvature in advance; then a laryngoscope 30 is adapted to depress the patient's tongue base so that an anaesthetist may put the endotracheal tube 20 and the endotracheal tube stylet 10a through the patient's mouth, where the endotracheal tube stylet 10a is withdrawn when the endotracheal tube 20 is inserted into the trachea.

Even though the usage of a stylet with an endotracheal tube may facilitate the intubation process, there are still some inconveniences in practice. For example, when applied in more difficult cases, such as patients with shorter necks or thicker tongues, the endotracheal tube is occasionally blocked by the patient's epiglottis cartilage and cannot be moved into the trachea because the shape of the stylet is not changeable. As shown in FIG. 1, due to the patient's protruding epiglottis cartilage, the endotracheal tube 20 cannot be installed easily.

In view of the fact that efficient intubation is critical for the survival of a patient, there is a need to provide an endotracheal tube stylet capable of changing the shape of the endotracheal tube. The endotracheal tube stylet shall have a changeable shape for redirecting the orientation of the endotracheal tube.

### SUMMARY OF THE INVENTION

It is therefore an object of the present invention to provide an endotracheal tube which enables a user to change the shape of the endotracheal tube during the intubation.

It is another object of the present invention to provide an endotracheal tube stylet insertable in an endotracheal tube. During the intubation, a user may move a patient's epiglottis cartilage with the endotracheal tube stylet.

It is still another object of the present invention to provide an endotracheal tube stylet made of relatively rigid material. The endotracheal tube stylet is capable of bearing a stronger force and changing its shape when bent. Moreover, the endotracheal tube stylet may be used to change the shape of the endotracheal tube from within the endotracheal tube; therefore, the endotracheal tube stylet is retained in the endotracheal tube and does not come in contact with the patient.

It is yet another object of the present invention to provide an endotracheal tube stylet which may be used with various devices, such as an illumination device, an image-capturing device, a light guide, etc., so as to increase the efficiency of the intubation.

It is also an object of the present invention to provide an endotracheal tube installation kit which, when assembled, may overcome the problems in difficult intubation cases.

To attain the object mentioned above, this invention provides an endotracheal tube stylet insertable into an endotracheal tube, the endotracheal tube stylet comprising: a first coupling member; a second coupling member coupled to the first coupling member; an operable member connected to the first or the second coupling member, the operable member being capable of changing the relative position of the first and the second coupling members; and a turnable member connected to the first or the second coupling member, wherein the turnable member may be redirected by the change of the relative position of the first and the second coupling members so as to change the shape of the endotracheal tube.

In some cases, the endotracheal tube stylet of this invention may selectively comprise a clasp member mountable on the first coupling member for retaining the turnable member in the endotracheal tube in use.

In addition, the present invention also provides an endotracheal tube installation kit comprising a laryngoscope, an endotracheal tube, and an endotracheal tube stylet insertable into the endotracheal tube, the endotracheal tube stylet comprising: a first coupling member; a second coupling member coupled to the first coupling member; an operable member connected to the first or the second coupling member, the operable member being capable of changing the relative position of the first and the second coupling members; and a turnable member connected to the first or the second coupling member, wherein the turnable member may be redirected by the change of the relative position of the first and the second coupling members so as to change the shape of the endotracheal tube.

Other objects, advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1 illustrates the instruments of prior arts used in an intubation process.

FIG. 2 is an illustration of one embodiment of the endotracheal tube stylet of this invention inserted in an endotracheal tube.

FIG. 3 is an illustration showing how one embodiment of the endotracheal tube stylet of this invention inserted in an endotracheal tube may work.

FIG. 4 is an illustration showing how another embodiment of the endotracheal tube stylet of this invention inserted in an endotracheal tube may work.

FIGS. 5 and 6 illustrate one embodiment of the endotracheal tube installation kit of this invention.

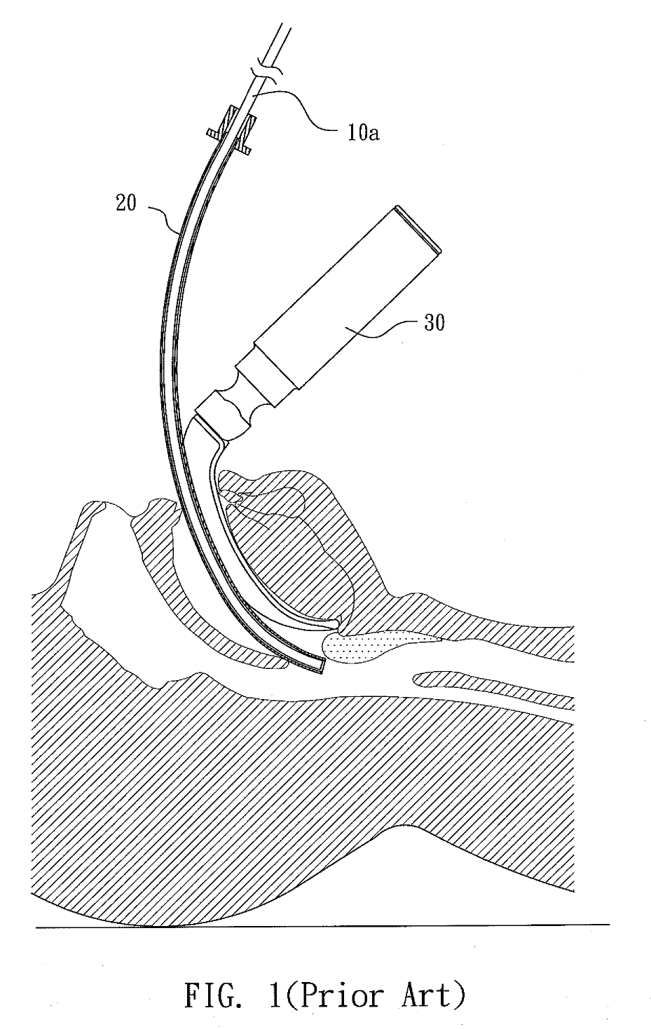

### DETAILED DESCRIPTION OF THE PREFERRED EMBODIMENT

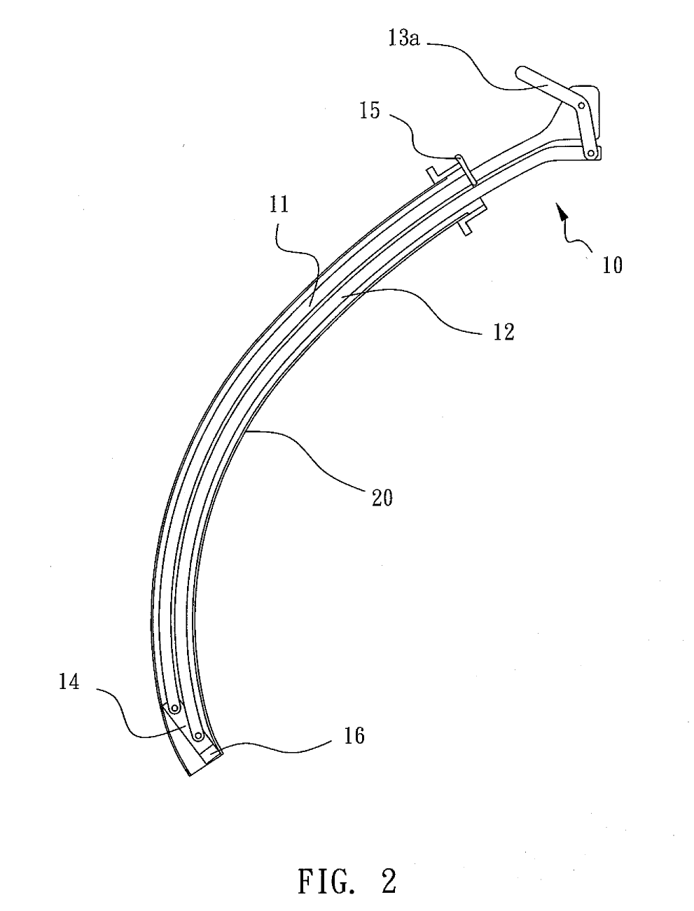

Please refer to FIG. 2 for an illustration of one embodiment of the endotracheal tube stylet 10 of this invention inserted in an endotracheal tube 20. As shown, the endotracheal tube stylet 10 of this invention mainly comprises: a first coupling member 11; a second coupling member 12 coupled to the first coupling member 11; an operable member 13a connected to the first or the second coupling members 11, 12, the operable member 13a being capable of changing the relative position of the first and the second coupling members 11, 12; and a turnable member 14 connected to the first or the second coupling members 11, 12, wherein the turnable member 14 may be redirected by the change of the relative position of the first and the second coupling members 11, 12 so as to change the shape of the endotracheal tube 20.

In this invention, the first coupling member 11 and the second coupling member 12 of the endotracheal tube stylet 10 may have an elongated and slightly curved shape. The material of these parts is not specifically limited; preferably, they are made of material allowing users to bend them to a desirable curvature. In most situations, the thicknesses of the two may be identical or different.

In some cases, the first coupling member 11, the second coupling member 12, the operable member 13a, and the turnable member 14 may be linked to each other in the same way as shown in FIG. 2, in which the first coupling member 11 and the second coupling member 12 are connected to the operable member 13a and the turnable member 14. However, the configuration is not limited thereto; any kinds of connections resulting in the turnable member 14 being controllable by the operable member 13a will suffice and fall within the scope of this invention.

For example, the operable member 13a may be connected to the first coupling member 11, and the turnable member 14 to the second coupling member 12; alternatively, the operable member 13a may be connected to the second coupling member 12, and the turnable member 14 to the first coupling member 11. The mechanical variation of the aforementioned members shall be obvious in the art and is omitted herein.

Meanwhile, the connections among the members are not specified; screwing, wrapping, and other connection means are all applicable. Preferably, without any limitation, the members are pivoted so that each member may be manipulated mutually.

In addition, the endotracheal tube stylet 10 may further comprise a clasp member 15 which may be mounted adjustably on the first coupling member 11 so as to keep the turnable member 14 in the endotracheal tube 20 in use. With the installment of the clasp member 15, a user may, in view of the length of the endotracheal tube 20, determine the length of the endotracheal tube stylet 10 in the endotracheal tube 20. Preferably, the endotracheal tube stylet 10 is disposed in the endotracheal tube 20 and does not protrude therefrom. Accordingly, direct contact between a patient and the endotracheal tube stylet 10 can be prevented, and the endotracheal tube 20 may be controlled to facilitate the intubation process. Also, the clasp member 15 may be mounted on the first coupling member 11 adjustably so that a user may adjust its position, allowing the endotracheal tube stylet 10 to protrude from the endotracheal tube 20.

Undoubtedly, the endotracheal tube stylet 10 of this invention may be used with different auxiliary devices so as to provide additional functions. The auxiliary device may include an optical fiber having one end connected to the turnable member 14 and the other end allowing the observation of the opening of the patient's trachea. The auxiliary device may also include an image-capturing device mounted on the turnable member 14 for capturing images of the opening of the patient's trachea; the images may be transmitted to a display installed internally on one end of the endotracheal tube stylet 10 adjacent to the operable member 13a or externally outside for displaying the images via a wire or wirelessly. Furthermore, the auxiliary device may also comprise an illumination device 16 as shown in FIG. 2 for illuminating the patient's upper airway and facilitating the intubation process. The illumination device 16 may, with limitation, comprise a light emitting diode light bulb.

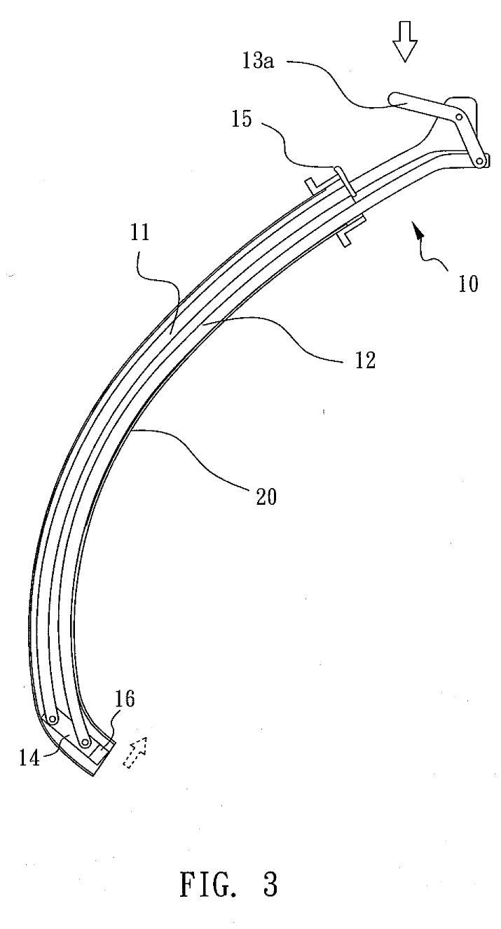

Please also refer to FIG. 3 for an illustration showing how one embodiment of the endotracheal tube stylet 10 of this invention inserted in an endotracheal tube 20 may work. When a user presses the operable member 13a, the first coupling member 11 and/or the second coupling member 12 may be driven and act upon the turnable member 14 disposed on one end of the operable member 13a, and the shape of the endotracheal tube 20 is changed thereby.

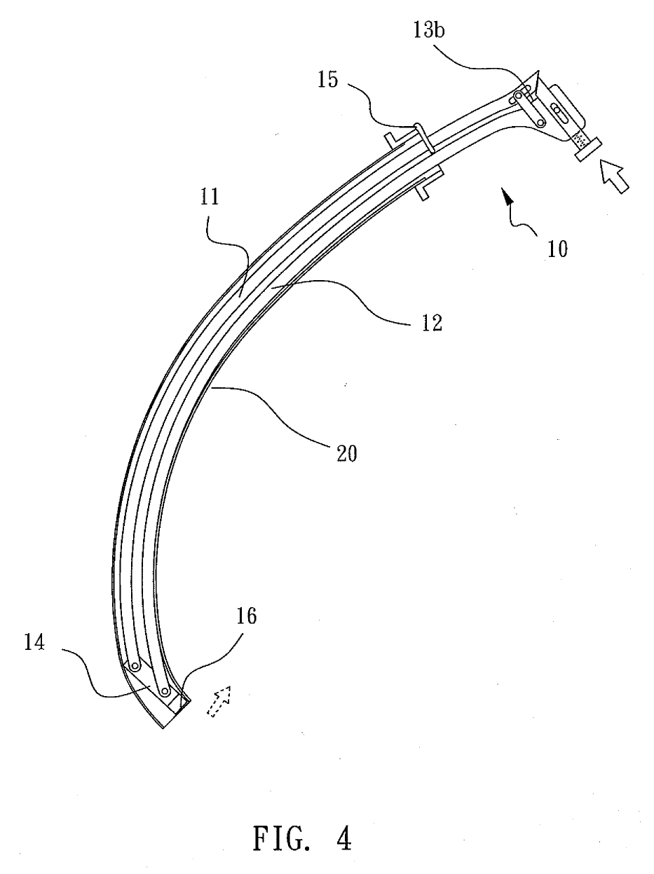

Refer to FIG. 4 for an illustration showing how another embodiment of the endotracheal tube stylet 10 of this invention inserted in an endotracheal tube 20 may work. In this embodiment, the operable member 13b has an oblique plane connected to the second coupling member 12; as a result, the second coupling member 12 may be moved by sliding the oblique plane of the operable member 13b; consequently, the first coupling member 11 and the turnable member 14 are moved correspondingly, and the shape of the endotracheal tube 20 is changed thereby.

It should be noted that even though only two different mechanisms, one achieved by the pivoting relation among members and the other by an oblique plane, are disclosed by this invention in FIGS. 2 and 4, the working mechanism of the members of the endotracheal tube stylet 10 of this invention is not limited thereto. Other means of connecting two coupling members and using an operable member to redirect a turnable member shall be within the spirit of the present invention. For example, the oblique plane mentioned above may also be applied to the interface between the first coupling member and/or the second coupling member.

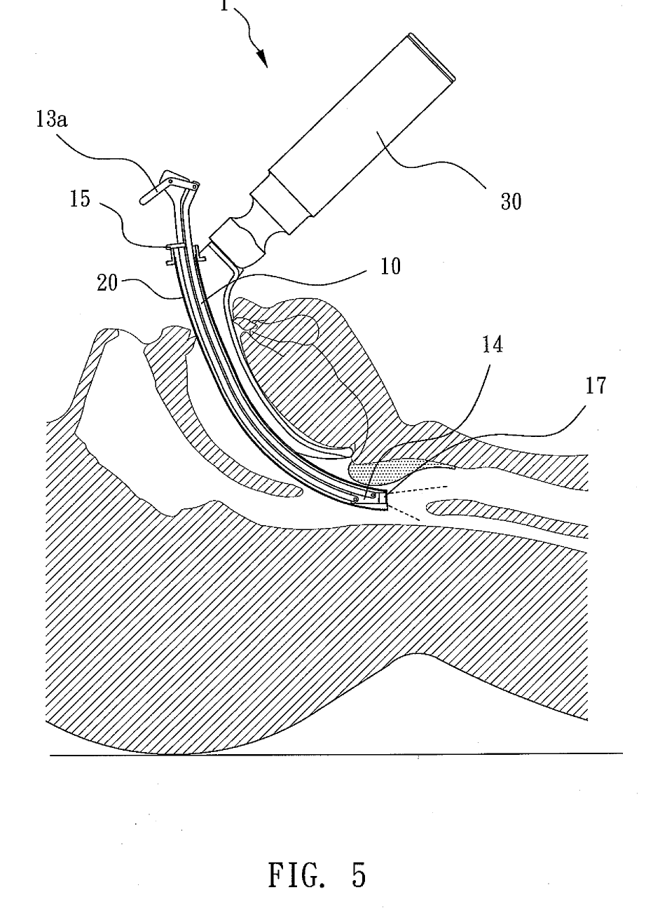

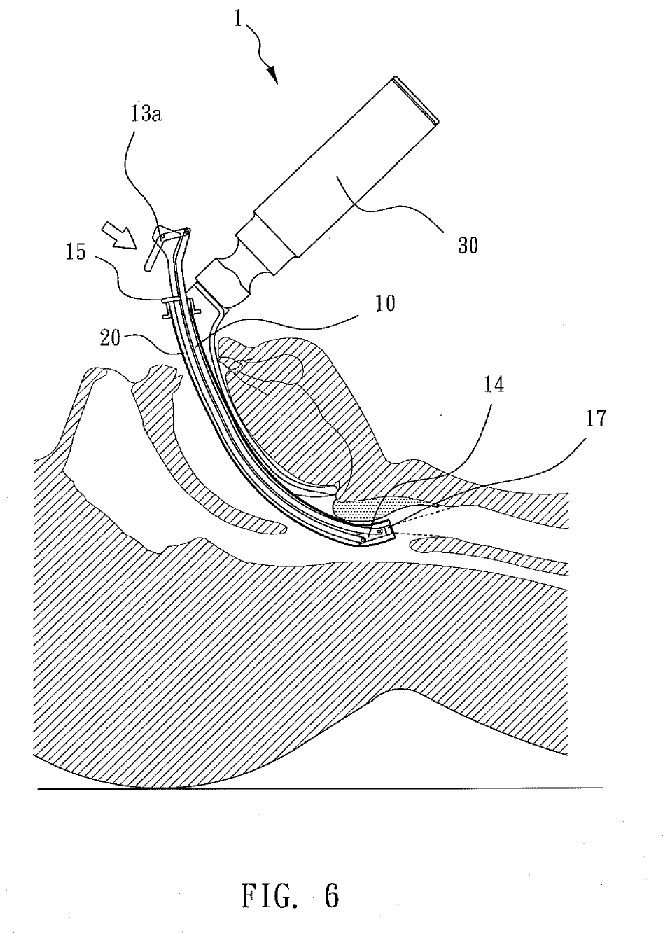

Finally, refer to FIGS. 5 and 6 for an illustration of one embodiment of the endotracheal tube installation kit 1 of this invention. The endotracheal tube installation kit 1 of this invention mainly comprises a laryngoscope 30, an endotracheal tube 20, and an endotracheal tube stylet 10 insertable into the endotracheal tube 20. The characteristics and components of the endotracheal tube stylet 10 are as described above, so further elaboration is omitted herein.

As shown in FIG. 5, when a user inserts the endotracheal tube 20 and the endotracheal tube stylet 10 wrapped therein into a patient's upper airway, occasionally the two instruments are blocked by the patient's epiglottis cartilage, making the intubation very difficult to carry out. The user may, as shown in FIG. 6, in light of the image captured by the image-capturing device 17 disposed on the turnable member 14, press the operable member 13a to move the turnable member 14 and thus to change the direction of the endotracheal tube 20 by slightly raising the epiglottis cartilage with the instruments. Accordingly, an angle suitable for the installation of the endotracheal tube 20 may be obtained, enabling the completion of the intubation.

Although the present invention has been explained in relation to its preferred embodiments, it is to be understood that many other possible modifications and variations can be made without departing from the spirit and scope of the invention as hereinafter claimed.

What is claimed is:

1. An endotracheal tube stylet insertable into an endotracheal tube, the endotracheal tube stylet comprising:
a first coupling member;
a second coupling member coupled to the first coupling member;
an operable member connected to the first or the second coupling member, the operable member being capable of changing the relative positions of the first and the second coupling members; and
a turnable member connected to the first or the second coupling member, wherein the turnable member may be redirected by the change of the relative position of the first and the second coupling members so as to change the shape of the endotracheal tube.

2. The endotracheal tube stylet as claimed in claim 1, wherein the operable member is connected to the first and the second coupling members.

3. The endotracheal tube stylet as claimed in claim 2, wherein the operable member is pivoted to the first and the second coupling members.

4. The endotracheal tube stylet as claimed in claim 1, wherein the turnable member is connected to the first and the second coupling members.

5. The endotracheal tube stylet as claimed in claim 4, wherein the turnable member is pivoted to the first and the second coupling members.

6. The endotracheal tube stylet as claimed in claim 1, further comprising a clasp member mounted on the first coupling member for retaining the turnable member in the endotracheal tube in use.

7. The endotracheal tube stylet as claimed in claim 6, wherein the clasp member is adjustably mounted on the first coupling member.

8. The endotracheal tube stylet as claimed in claim 1, further comprising an illumination device connected to the turnable member.

9. The endotracheal tube stylet as claimed in claim 8, wherein the illumination device comprises a light emitting diode light bulb.

10. The endotracheal tube stylet as claimed in claim 1, further comprising an image-capturing device connected to the tamable member.

11. The endotracheal tube stylet as claimed in claim 1, further comprising a light guide connected to the turnable member.

12. The endotracheal tube stylet as claimed in claim 11, wherein the light guide comprises an optical fiber.

13. An endotracheal tube installation kit comprising a laryngoscope, an endotracheal tube, and an endotracheal tube stylet insertable into the endotracheal tube, the endotracheal tube stylet comprising:
a first coupling member;
a second coupling member coupled to the first coupling member;
an operable member connected to the first or the second coupling member, the operable member being capable of changing the relative position of the first and the second coupling members; and
a turnable member connected to the first or the second coupling member, wherein the turnable member may be redirected by the change of the relative position of the first and the second coupling members so as to change the shape of the endotracheal tube.

14. The endotracheal tube installation kit as claimed in claim 13, wherein the endotracheal tube stylet further comprises a clasp member adjustably mounted on the first coupling member for retaining the turnable member in the endotracheal tube in use.

15. The endotracheal tube installation kit as claimed in claim 13, wherein the endotracheal tube stylet further comprises a light emitting diode light bulb connected to the turnable member.

16. The endotracheal tube installation kit as claimed in claim 13, wherein the endotracheal tube stylet further comprises an image-capturing device connected to the turnable member.

17. The endotracheal tube installation kit as claimed in claim 13, wherein the endotracheal tube stylet further comprises a light guide connected to the turnable member.

18. The endotracheal tube installation kit as claimed in claim 13, wherein the operable member is pivoted to the first and the second coupling members.

19. The endotracheal tube installation kit as claimed in claim 13, wherein the turnable member is pivoted to the first and the second coupling members.
